(19) United States
(12) Patent Application Publication
(10) Pub. No.: US 2004/0267328 A1
(43) Pub. Date: Dec. 30, 2004

(54) Electrode selection system for medical electrical leads

(75) Inventors: Edwin G. Duffin, North Oaks, MN (US); Thomas D. Brostrom, Lindstrom, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/601,476

(22) Filed: Jun. 24, 2003

(51) Int. Cl. A61N 1/375

(52) U.S. Cl. 607/37

(57) ABSTRACT

A lead electrode, from an array of lead electrodes, is selectively coupled to a pulse generator device by positioning a lead connector including an array of connector contacts corresponding to the array of lead electrodes within a connector bore of the device for electrical engagement of a selected connector contact from the array of connector contacts with a device contact; the selected connector contact corresponding to the selected electrode.

### FIELD OF THE INVENTION

Embodiments of the present invention relate to implantable medical device connectors and more particularly to a connection system adapted to allow selection of an electrode from a plurality of electrodes included on a medical electrical lead for permanent connection with the medical device.

### BACKGROUND OF THE INVENTION

Implantable medical electrical stimulation and/or sensing leads are well known in the fields of cardiac stimulation and monitoring, including cardiac pacing and cardioversion/defibrillation, and in other fields of electrical stimulation or monitoring of electrical signals or other physiologic parameters. In the field of cardiac stimulation and monitoring, endocardial leads are placed through a transvenous route to locate one or more stimulation and/or sense electrodes, along or at the distal end of the lead body, in a desired location within a chamber of the heart or within a blood vessel of the heart. Epicardial leads are routed from a subcutaneous site to dispose one or more stimulation and/or sense electrodes, along or at the distal end of the lead body, at an epicardial site on the heart. An implantable pulse generator (IPG), pacemaker or cardioverter/defibrillator, or a monitor is coupled to the heart through one or more of such endocardial or epicardial leads. Means for implanting such cardiac leads are known to those skilled in the art of pacing and defibrillation therapy.

Proximal ends of such cardiac leads typically are formed with a lead connector assembly that is inserted into a bore of a connector header of the IPG or monitor. The lead body extending distally from the connector assembly typically includes one or more insulated conductors surrounded by an outer insulative sheath. Each conductor couples a lead connector contact of the lead connector assembly with a distal stimulation and/or sense electrode.

More recently, medical electrical leads have been constructed with an array of pacing and/or sensing electrodes from which one or more electrodes may be selected to optimize electrical stimulation therapy and/or monitoring. Examples of such leads include: a coronary vein lead implanted to stimulate a left atrium or left ventricle or both; a right atrial or right ventricular lead implanted to sense and/or stimulate a right side of a heart; a single pass lead implanted in a right atrium and a right ventricle to provide dual chamber therapy; and a physiological lead implanted to stimulate and/or sense directly a portion of the cardiac conduction system. Additionally epicardial leads, leads adapted for deep brain stimulation, and other leads adapted to stimulate other muscles of the body may include an array of electrodes from which one or more electrodes may be selected to optimize therapy. A connection system for these types of leads needs to be adapted for the selection of one or more electrodes included in the array.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a schematic diagram of an exemplary implantable medical system in accordance with embodiments of the present invention;

FIG. 2A is a plan view of an exemplary electrical lead according to one embodiment of the present invention;

FIG. 2B is a radial cross-section through section line A-A of the lead shown in FIG. 2A;

FIG. 2C is a schematic diagram of a distal portion of the lead shown in FIG. 2A implanted in a coronary vein;

FIGS. 3A-E are partial section views of the lead connector shown in FIG. 2A positioned at multiple points within a device header bore for selection of a pair of electrodes;

FIG. 4 is a schematic diagram of an alternate embodiment of a lead implanted in a coronary vein;

FIG. 5 is a schematic diagram of yet another embodiment of a lead implanted in a right atrium;

FIGS. 6A-D are partial section views of the lead connector shown in FIGS. 4-5 positioned at multiple points within a device header bore for selection of a single electrode;

FIG. 7A is a schematic diagram of another alternate embodiment of a lead implanted in a right ventricle;

FIG. 7B is a schematic diagram of the lead illustrated in FIG. 7A implanted in a right atrium;

FIGS. 8A-B are partial section views of the lead connector of the lead illustrated in FIGS. 7A-B positioned at multiple points within a device header bore for selection of a single electrode;

FIG. 8C is a partial section view of a lead connector positioned in a device header bore according to an alternate embodiment of the present invention;

FIGS. 9A-B are partial section views of a lead connector positioned at multiple points in a device header bore according to yet another embodiment of the present invention;

FIGS. 9A-B are partial section views of lead connector, a connector header and an insertion tool in part relation according to an alternate embodiment of the present invention;

FIG. 10 is an enlarged detail view of an interface between device contact and a connector contact according to one embodiment of the present invention;

FIG. 11 is an enlarged detail view of an alternate interface providing reversible locking according to the present invention;

FIG. 12 is a partial section view of a lead connector positioned in a device header bore including yet another embodiment of an interface providing reversible locking; and

FIG. 13 is a partial section view of a lead connector positioned in a connector bore according to an alternate embodiment including an actuated member for reversibly locking the connector in the bore.

### DETAILED DESCRIPTION OF EMBODIMENTS OF THE INVENTION

The following detailed description of the invention is merely exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

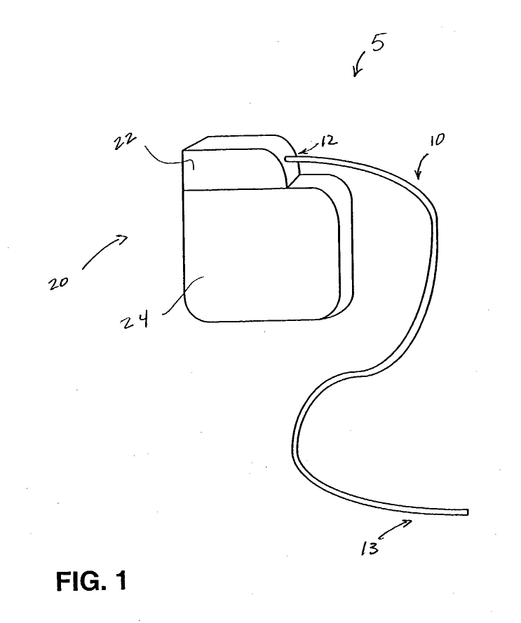

FIG. 1 is a schematic diagram of an exemplary implantable medical system 5 in accordance with the present invention. FIG. 1 illustrates medical system 5 including a pulse generator device 20 and an implantable electrical lead 10 mechanically and electrically coupled to the device 20 via a connector header 22. Device 20 includes an enclosure 24, containing a battery and electronic circuitry and other components, to which connector header 22 is attached. Connector header 22 includes a connector bore (not shown) into which a connector (not shown) terminating a proximal end 12 of lead 10 is inserted. One or more electrical device contacts within the bore are electrically connected to the electronic circuitry via feedthrough pins of feedthroughs (not shown) mounted to extend through hermetically sealed enclosure 24. According to the present invention, the lead connector is positioned within the bore of header 22 such that the one or more electrical device contacts couple with one or more selected connector contacts which correspond to selected electrodes (not shown) joined to a distal portion 13 of lead 10; multiple embodiments of system 5 are described in conjunction with the following Figures.

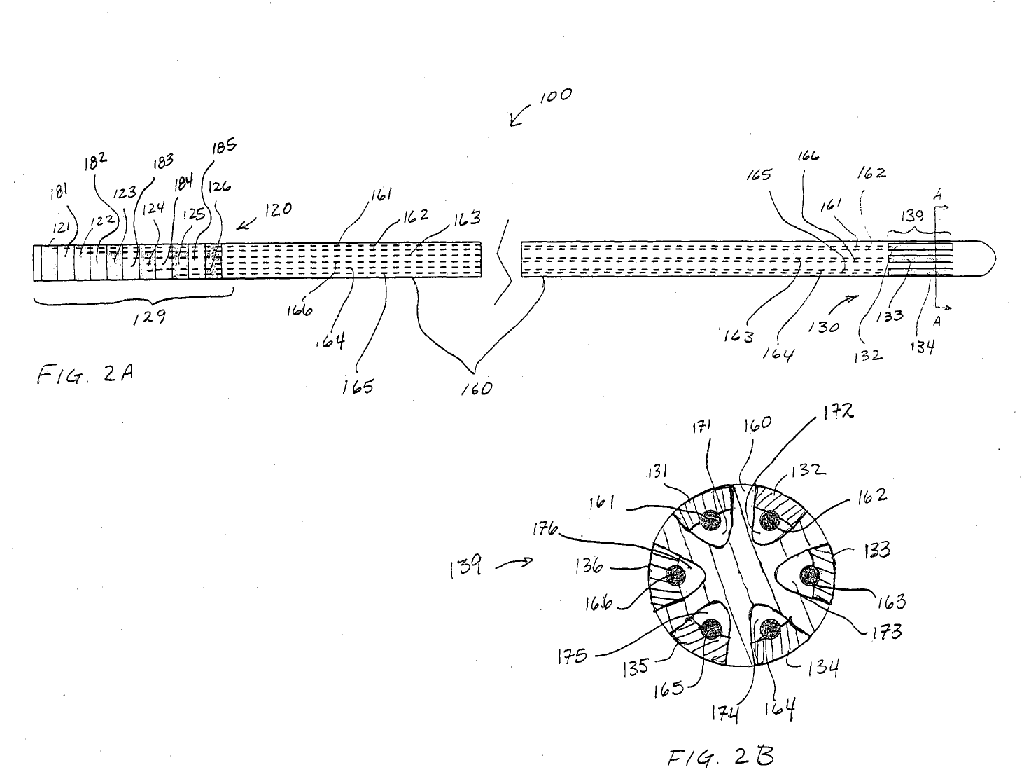

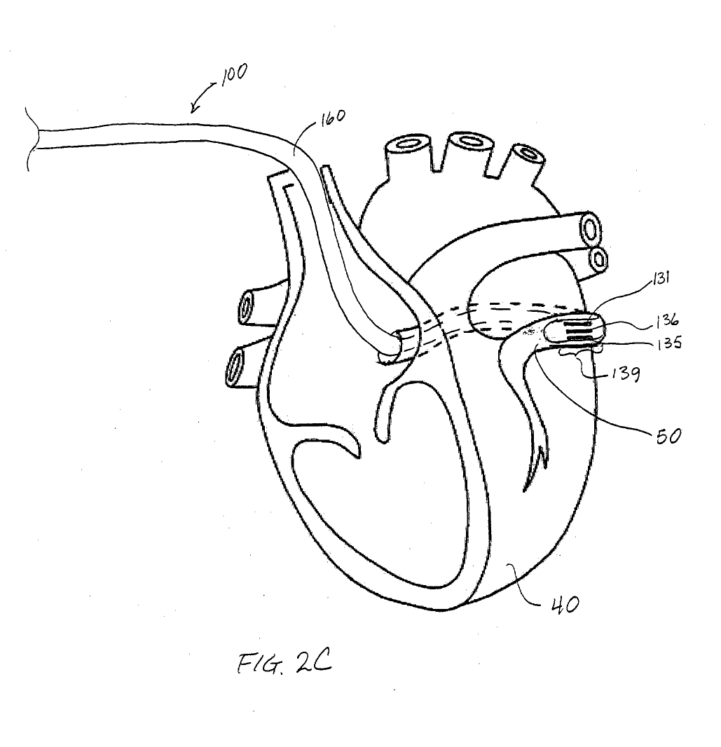

FIG. 2A is a plan view of an exemplary electrical lead 100 according to one embodiment of the present invention; FIG. 2B is a radial cross-section through section line A-A of lead 100; and FIG. 2C is a schematic diagram of a distal portion of lead 100 implanted in a coronary vein. FIG. 2A illustrates lead 100 including a lead body 160 terminated at a proximal end 120 by a connector 129; connector 129 includes connector contacts 121, 122, 123, 124, 125, and 126 each electrically isolated from one another by insulative spacers 181, 182, 183, 184, and 185 and each joined to an electrode of a circumferential electrode array 139 via elongated conductors 161, 162, 163, 164, 165, and 166, respectively, shown as dashed lines, extending through lead body 160. Each of conductors 161-166, in accordance with embodiments of the present invention, are composed of a material capable of reliably conducting electrical current after having been subjected to repeated bending and torsion loads, imposed by an implant environment, for example an MP35N alloy, and may be a multi-wire cable, a coil, including one or more wires, or a single wire of a multi-filar coil, or other types of conductors known for use in medical electrical leads to those skilled in the art.

As illustrated in FIG. 2B circumferential array 139 includes electrodes 131, 132, 133, 134, 135 and 136 and lead body 160 includes lumens 171, 172, 173, 174, 175, and 176, through which conductors 161-166 extend. In various embodiments, conductors 161-166 are mechanically and electrically coupled to connector contacts 121-126 and to electrodes 131-136 by welds, crimps or stakes, methods for forming and configurations of which are well known to those skilled in the art of lead construction; furthermore, lead body 160 and insulative spacers 181-185 are composed of a durable biocompatible and biostable insulative polymer, examples of which include silicone or polyurethane. Electrodes 132-136, in some embodiments, are formed from a platinum alloy and may have a porous surface structure; alternative and or additional materials from which electrodes 132-136 may be formed include palladium, titanium, tantalum, rhodium, iridium, carbon, vitreous carbon and alloys, oxides and nitrides of such metals or other conductive or even semi-conductive materials. Examples of acceptable electrode materials and associated fabrication techniques employed to achieve a porous surface structure are well known to those skilled in the art.

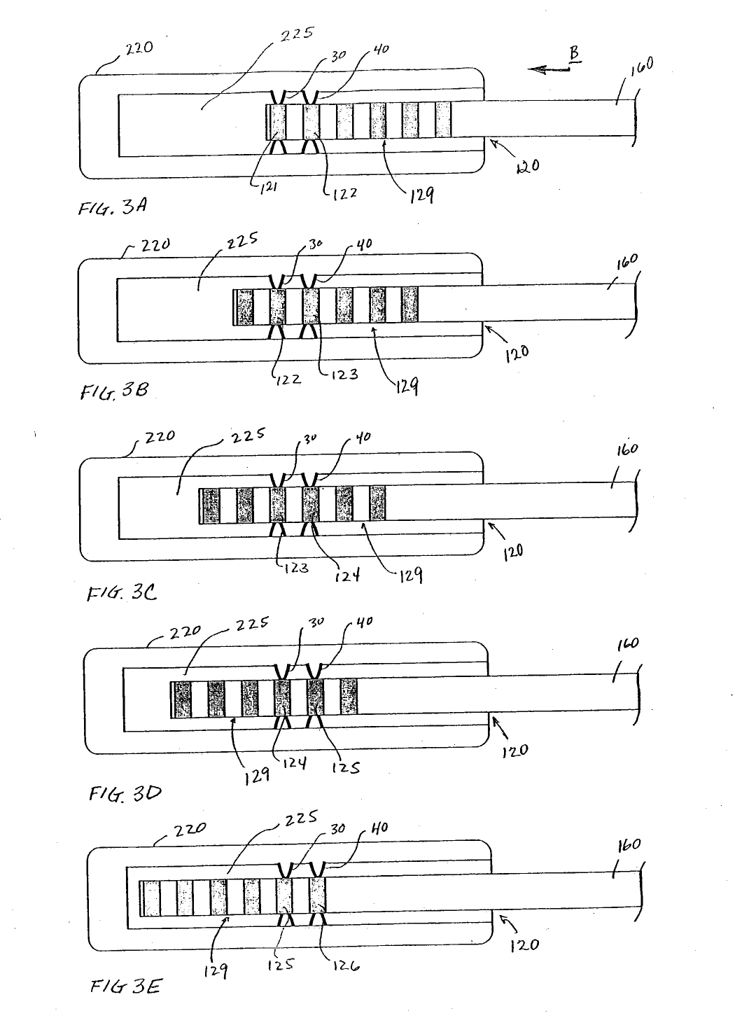

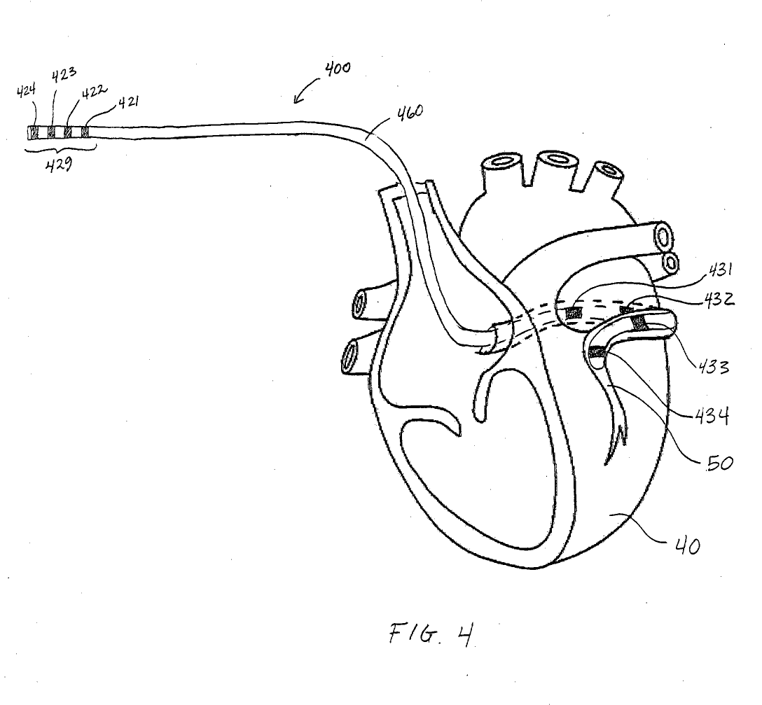

According to embodiments of the present invention lead 100 is implanted in a coronary vein 50 of a heart 40, as illustrated in FIG. 2C, wherein one or two electrodes of array 139 are selected to deliver pacing stimulation from a pulse generator device such as device 20 illustrated in FIG. 1; selection may be based on a reduced cardiac stimulation threshold and, or prevention of phrenic nerve stimulation. For example, in FIG. 2C electrodes 131, 135, and 136 are shown directed away from a surface heart 40, therefore one or two of electrodes 132, 133, and 134 would likely be selected to deliver pacing stimulation, since one or two of these electrodes are directed more toward the surface of heart 40. According to embodiments of the present invention, a selection of one or two electrodes of array 139 is accomplished by positioning connector 129 in a bore of a device connector header, such as header 22 illustrated in FIG. 1, so that one or two corresponding contacts of connector 129 are electrically engaged by one or two device contacts. For example, if electrode 132 is selected, lead connector contact 122 is positioned for electrical engagement within a header bore; alternatively as is further illustrated in FIGS. 3A-E, if two electrodes are selected, a pair of lead connector contacts are positioned for electrical engagement in a header bore.

FIGS. 3A-E are partial section views of lead connector 129 positioned at multiple points within a device header bore 225 for selection of a pair of electrodes. FIGS. 3A-E illustrate a header 220 including bore 225 wherein a first device contact 30 and second device contact 40 are mounted for electrical engagement of a pair of lead connector contacts corresponding to a selected pair of lead electrodes. According to embodiments of the present invention, a connector, for example connector 129, may be positioned and reversibly locked in bore 225 for electrical coupling with device contacts 30 and 40 as illustrated in any one of FIGS. 3A-E: FIG. 3A illustrating connector contacts 121 and 122 electrically engaged for selection of electrodes 131 and 132; FIG. 3B illustrating connector contacts 122 and 123 electrically engaged for selection of electrodes 132 and 133; FIG. 3C illustrating connector contacts 123 and 124 electrically engaged for selection of electrodes 133 and 134; FIG. 3D illustrating connector contacts 124 and 125 electrically engaged for selection of electrodes 134 and 135; and FIG. 3E illustrating connector contacts 125 and 126 electrically engaged for selection of electrodes 135 and 136.

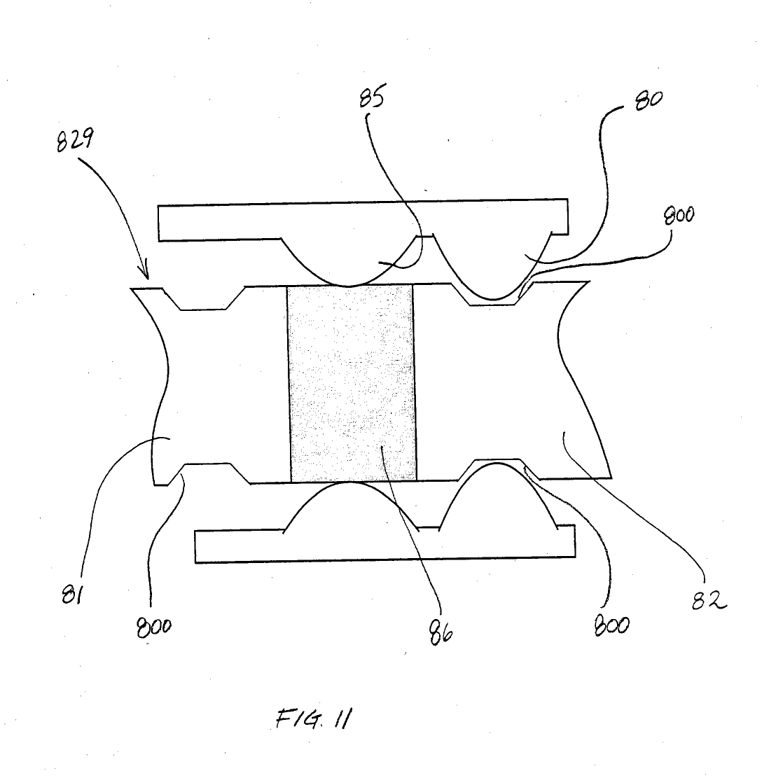

Alternate means for reversibly locking a connector in a bore, for example connector 129 in bore 225, will be described herein below in conjunction with FIGS. 10A-12B. Alternate means for positioning a connector in a bore include insertion into bore per an arrow B illustrated in FIG. 3A and pressing or laying a connector into a bore from a direction perpendicular to arrow B via a slot or an opening along a length of the bore, which is subsequently sealed off. Alternate embodiments of device contacts, for example first and second device contacts 30 and 40, include set-screws, springs, such as a garter spring or an assembly of inwardly extending force beams, and other types of electrical contacts well known to those skilled in the art.

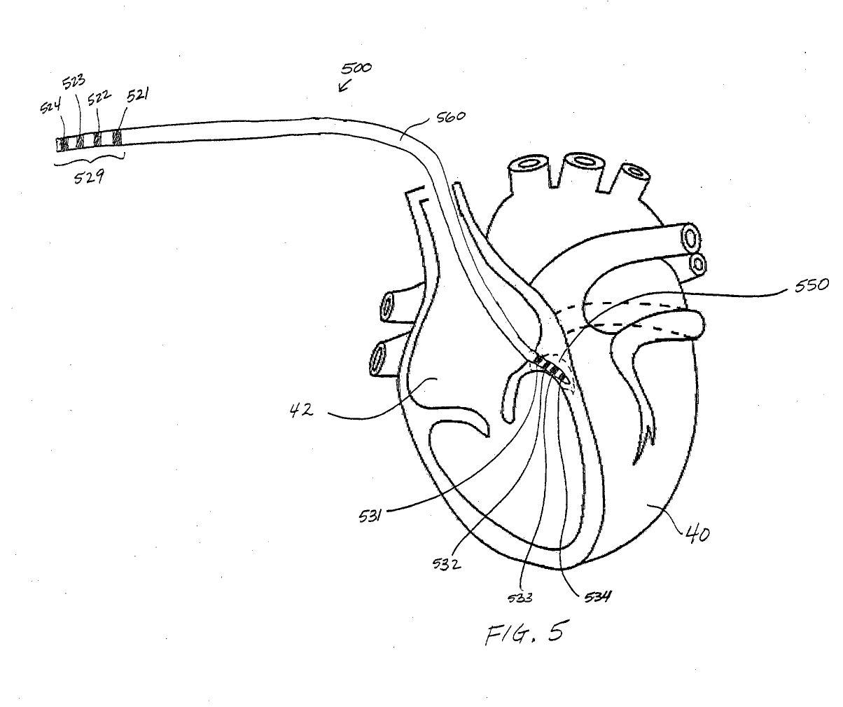

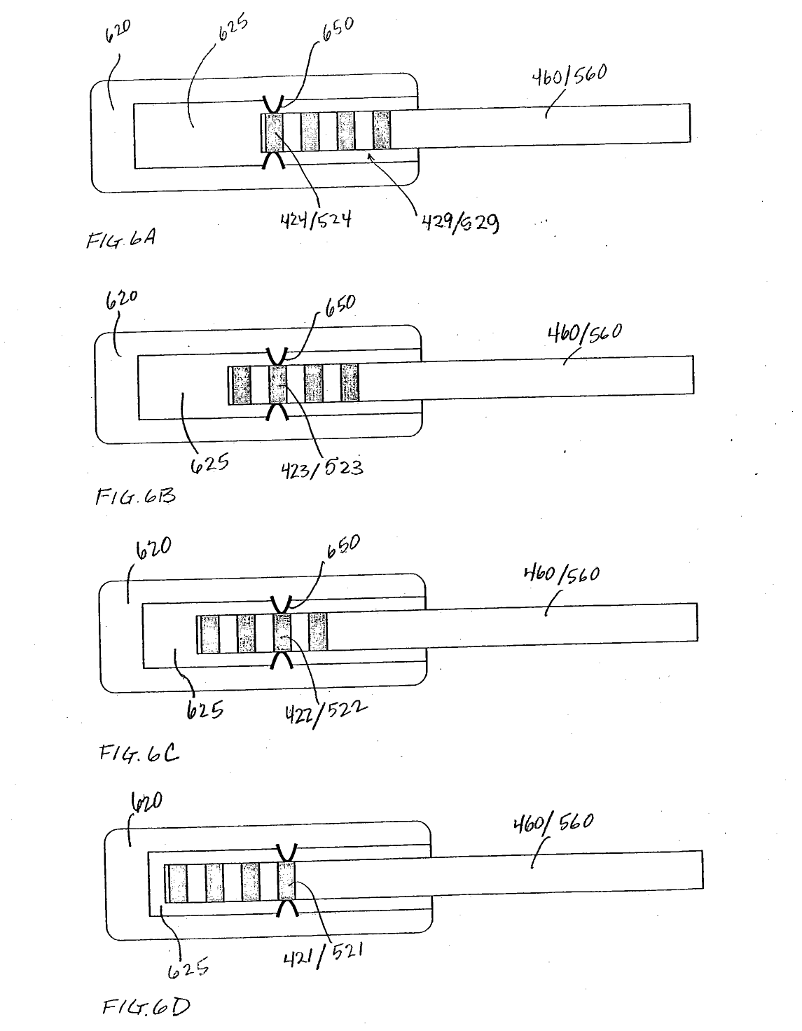

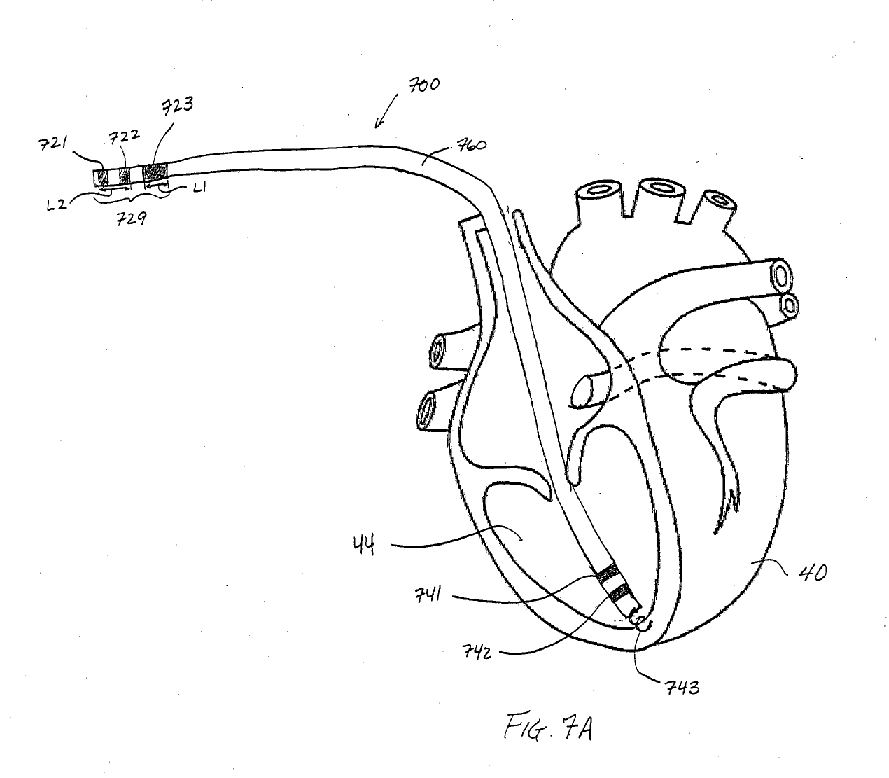

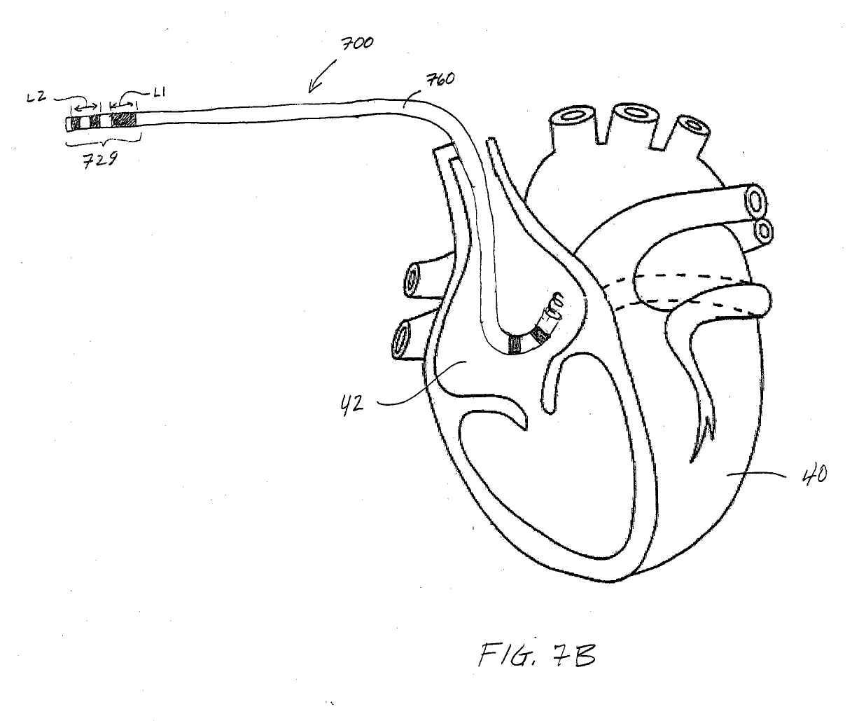

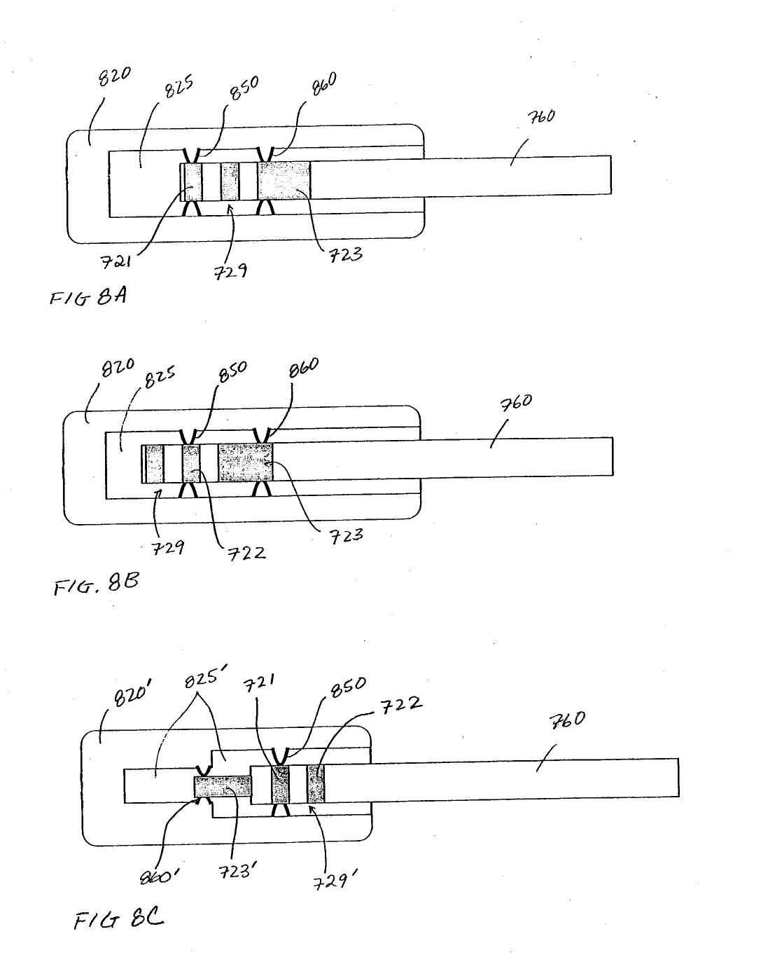

FIG. 4 is a schematic diagram of an alternate embodiment of a lead 400 implanted in coronary vein 50; and FIG. 5 is a schematic diagram of yet another embodiment of a lead 500 implanted in a right atrium 42. FIG. 4 illustrates lead 400 including a lead body 460 terminated at a proximal end by a connector 429; connector 429 includes connector contacts 424, 423, 422, and 421 each joined to electrodes 434, 433, 432, and 431, formed in a linear array at a distal end of lead body 460, by elongated insulated conductors, not shown but similar to those previously described in conjunction with FIG. 2A. FIG. 5 illustrates lead 500 including a lead body 560 terminated at a proximal end by a connector 529; connector 529 includes connector contacts 524, 523, 522, and 521 each joined to electrodes 534, 533, 532, and 531, formed in a linear array at a distal end of lead body 560, by elongated insulated conductors, not shown but similar to those previously described in conjunction with FIG. 2A. One or more of electrodes 434, 433, 432, and 431 of lead 400 may be selected for stimulation of a left side of heart 40 from coronary vein 50 in a manner similar to that previously described according to the present invention; likewise, one or more of electrodes 534, 533, 532, and 531 of lead 500 may be selected for direct stimulation of a portion of the conduction system of heart 40, for example a bundle of His 550 as illustrated in FIG. 5. According to some embodiments of the present invention a single electrode from the plurality of electrodes of either lead 400 or 500 is selected as illustrated in FIGS. 6A-D. Although only lead 400 and 500 are shown in FIGS. 4 and 5, respectively, it is recognized that additional leads may be implanted in heart 40, which leads including one or more electrodes to operate in conjunction with the one or more selected electrodes of leads 400 and 500. A selection of electrodes on either of leads 400 and 500 may be based a location of each electrode at an implant site with respect to a target site in the cardiac tissue.

FIGS. 6A-D are partial section views of the lead connectors shown in FIGS. 4-5 positioned at multiple points within a device header bore for selection of a single electrode. FIGS. 6A-D illustrate a header 620 including bore 625 wherein a device contact 650 is mounted for electrical engagement of a lead connector contact corresponding to a selected lead electrode. According to embodiments of the present invention, a connector, for example connector 429 of lead 400 or connector 529 of lead 500, may be positioned and reversibly locked in bore 625 for electrical coupling with device contact 650 as illustrated in any one of FIGS. 6A-D: FIG. 6A illustrating connector contact 424/524 electrically engaged for selection of electrode 434/534; FIG. 3B illustrating connector contacts 423/523 electrically engaged for selection of electrode 433/533; FIG. 3C illustrating connector contact 422/522 electrically engaged for selection of electrode 432/532; FIG. 3D illustrating connector contacts 421/521 electrically engaged for selection of electrode 431/531. Alternate means for positioning connector 429/529 in bore 625 and alternate embodiments of contact 650 are similar to those described in conjunction with FIGS. 3A-E.

FIG. 7A is a schematic diagram of another alternate embodiment of a lead 700 implanted in a right ventricle; and FIG. 7B is a schematic diagram of lead 700 implanted in right atrium 42. FIGS. 7A-B illustrate lead 700 including a lead body 760 terminated at a proximal end by a connector 729; connector 729 includes a connector contact array, including connector contacts 721 and 722 joined to electrodes 741 and 742, respectively, via elongated insulated conductors (not shown), and a third contact 723 joined similarly to a tip electrode 743. Tip electrode 743, formed at a distal tip of lead body 760, is illustrated as a helix, providing fixation of distal tip; according to embodiments of the present invention one of electrodes 741 and 742 is selected to operate in conjunction with tip electrode 743 as a bipolar pair, as is illustrated in FIGS. 8A-B. The selection of an electrode from electrodes 741 and 742 may be based on the chamber in which lead 700 is implanted for operation, for example, if lead 700 is implanted in right ventricle 44, as illustrated in FIG. 7A, electrode 741 may be selected for a larger gap between electrode 741 and tip electrode 743, while if lead 700 is implanted in right atrium 42, as illustrated in FIG. 7B, electrode 742 may be selected for a smaller gap between electrode 742 and tip electrode 743. In any case, selection of an electrode from electrodes 741 and 742 would typically be based upon desired sensing characteristics of the bipolar pair understood by those skilled in the art.

FIGS. 8A-B are partial section views of lead connector 729 positioned at multiple points within a device header bore 825 for selection of a single electrode from electrodes 741 and 742 to operate in conjunction with electrode 743 of lead 700 illustrated in FIGS. 7A-B. FIGS. 8A-B illustrate a header 720 including bore 725 wherein a first device contact 850 is mounted for electrical engagement of one of lead connector contacts 721, 722 while a second device contact 860 is mounted for electrical engagement of third connector contact 723 when either one of connector contacts 721, 722 is selected. As is illustrated in FIGS. 7A-B, third connector contact 723, corresponding to helix electrode 743, has a length L1 approximately equal to a length L2 of the array including contacts 721 and 722 so that when either 721 or 722 is positioned in header bore 825 for electrical engagement with first device contact 850, third connector contact 723 is electrically engaged by second device contact 860.

FIG. 8C is a partial section view of a lead connector 729′ positioned in a device header bore 825 illustrating an alternate embodiment wherein a third lengthened connector contact 723′, corresponding with tip electrode 743 (FIGS. 7A-B) is positioned proximal to the array including connector contacts 721, 722 and in a form of a reduced diameter terminal pin; likewise a second device contact 860′ is positioned proximal to first device contact 850 in a reduced diameter bore to accommodate third connector contact 723′.

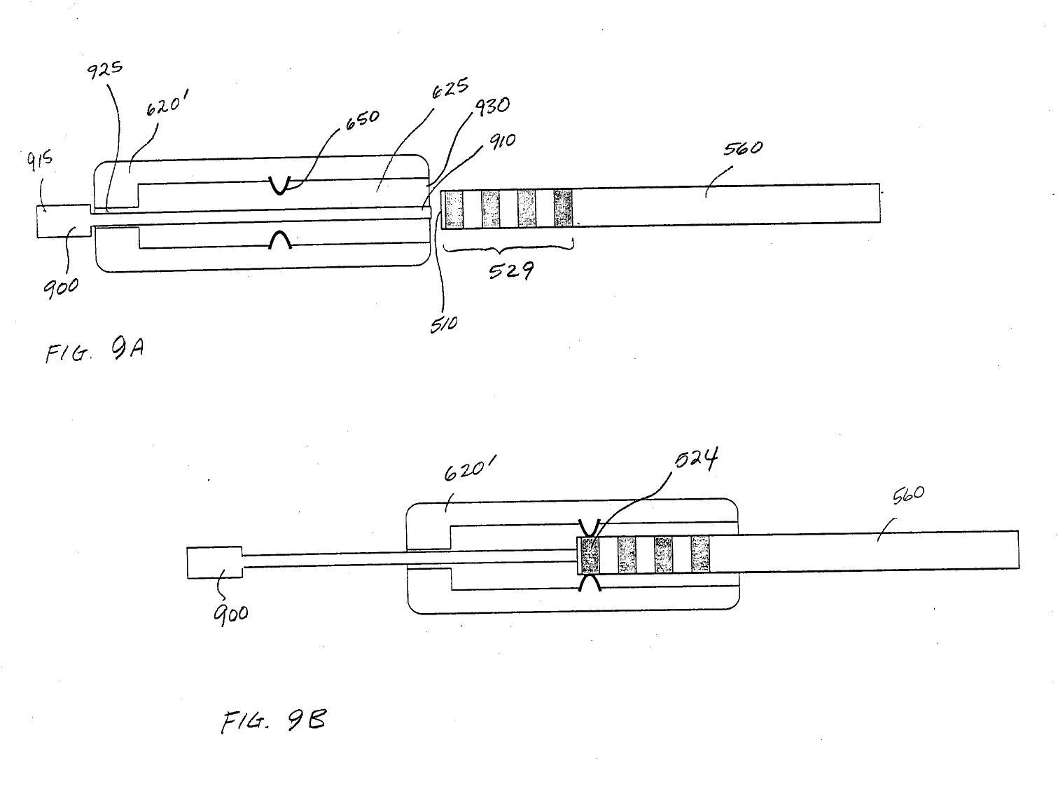

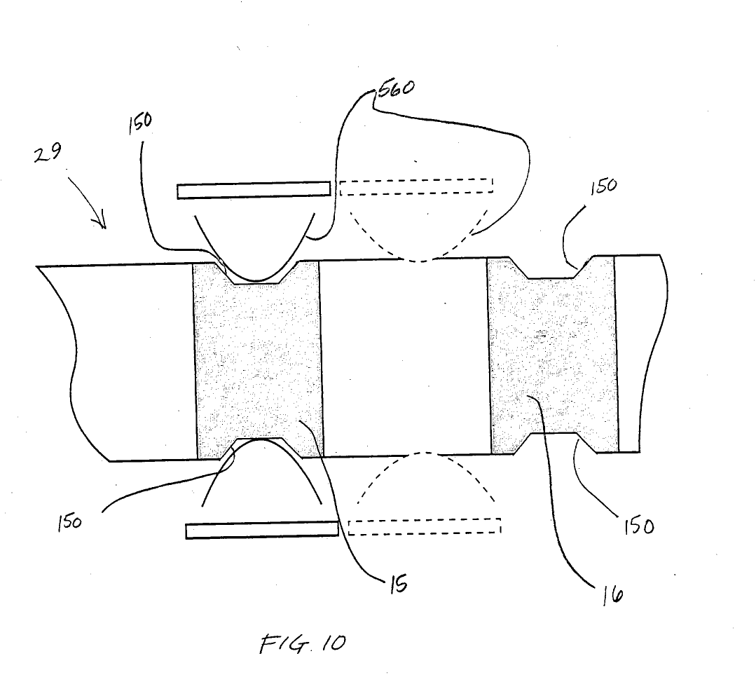

FIGS. 9A-B are partial section views of lead connector 529, a connector header 620′ and an insertion tool 900 in part relation according to an alternate embodiment of the present invention. FIGS. 9A-B illustrate one means by which a lead connector may be positioned within a connector header bore, such as connector 529 within bore 625, wherein header 620′ further includes a proximal opening 925 through which insertion tool 900, including a proximal handle 915 and a distal end 910 adapted for engaging a proximal end 510 of connector 529, may be inserted in order to pull connector 529 into bore 625 from a distal opening 930. According to embodiments of the present invention, once pull tool 900 has pulled connector 929 into position for engagement of a selected connector contact with device contact 650, for example connector contact 524 illustrated in FIG. 9B, pull tool 900 may be disengaged from proximal end 510 and removed through proximal opening 925. A grommet may be provided just inside opening 925 in order to seal bore 625 from bodily fluids after pull tool 900 has been removed. (Although not shown, additional means for sealing bore 625, and any other header bore described herein, from fluid ingress at a distal opening, for example opening 930, include seals within the bore adapted to sealing engage the lead connector and seals positioned about portions of lead connector adapted to sealingly engage an inner surface of the bore; either of which means is well known to those skilled in the art.) Pull tool 900 and method for use with a connector header and lead connector may correspond generally to that disclosed in U.S. Pat. No. 5,843,141 issued to Bischoff, et al., incorporated herein by reference in its entirety.

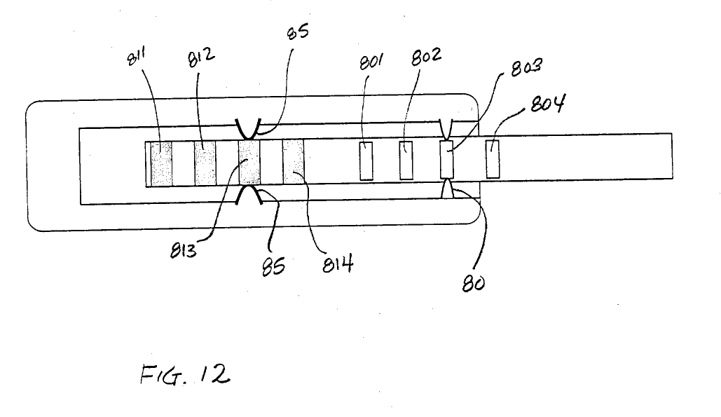

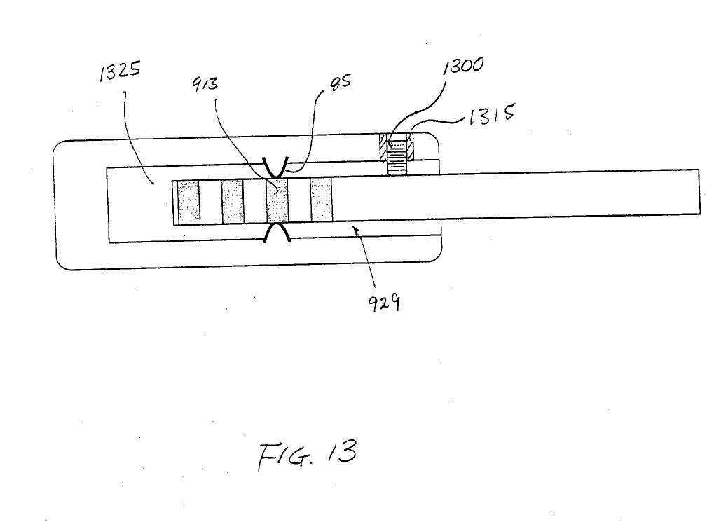

FIG. 10 is an enlarged detail view of an interface between a device contact 560 and a connector contact 15, wherein the interface provides reversible locking of a lead connector 29 within a connector header bore according to one embodiment of the present invention. FIG. 10 illustrates device contact 560 with solid lines as contact 560 is engaged within a surface depression 150 of connector contact 15, and with dashed lines as contact 560 is depressed/deflected when connector 29 is repositioned for subsequent engagement of contact 560 within another connector contact 16; thus, according to this embodiment each connector contact includes a surface depression 150 in which a device contact may be engaged for reversible locking of a connector in a bore. FIGS. 11-12B illustrate alternate means for reversible locking employing an element separate from a device contact.

FIG. 11 is an enlarged detail view of an alternate interface providing reversible locking within a connector header bore according to the present invention, wherein a deflectable member 80 is included to mate with surface depressions 800 formed in insulating spacers 81, 82 of a connector 829. Such insulating spacers were previously described in conjunction with FIG. 2A (spacers 181-185). Deflectable member 80, formed from a resilient material, may also serve to sealingly engage insulating spacers 81, 82. FIG. 11 illustrates connector 829 reversibly locked, via member 80 engaged in depression 800, for electrical engagement of a connector contact 86 by a device contact 85.

FIG. 12 is a partial section view of a lead connector 829′ positioned in a device header bore including yet another embodiment of an interface providing reversible locking. FIG. 12 illustrates deflectable member 80 positioned apart from device contact 85 to engage each in an array of surface depressions, 801, 802, 803 and 804 corresponding to each in an array of connector contacts 811, 812, 813, and 814, respectively; rather than being formed in spacers interspersed among device contacts 811-814, as is illustrated in the example of FIG. 11, array of depressions 801-804 are formed distal to array of connector contacts 811-814. FIG. 12 further illustrates 829′ reversibly locked, via member 80 engaged in depression 803, for electrical engagement of connector contact 813 by device contact 85. Further alternate embodiments employ an actuated member for reversibly locking of a lead connector that does not require surface depressions in the lead connector; examples of such embodiments include a set-screw, well known to those skilled in the art and illustrated in FIG. 13, and compression members exemplified in commonly assigned U.S. Pat. Nos. 5,766,042 and 6,080,188, the teachings of which are herein incorporated. FIG. 13 is a partial section view of a lead connector 929 positioned in a connector bore 1325, wherein a set-screw 1300 comprises an actuated member for reversibly locking the connector 929 in the bore 1325. FIG. 13 illustrates set-screw 1300 having been tightened down onto lead connector 929 through a threaded connector block 1315 to lock lead connector 929 in place for electrical engagement of connector contact 913 with device contact 85.

While specific embodiments have been presented in the foregoing detailed description of the invention, it should be clear that a vast number of variations exist. It should also be appreciated that the exemplary embodiments are only examples, and are not intended to limit the scope, applicability, or configuration of the invention in any way. Rather, the foregoing detailed description will provide those skilled in the art with a convenient road-map for implementing an exemplary embodiment of the invention. It should be understood that various changes may be made in the function and arrangement of elements described in an exemplary embodiments without departing from the scope of the invention as set forth in the appended claims.

What is claimed is:

1. A medical system, comprising:
an array of lead electrodes including a selected electrode;
an assembly of elongated insulated conductors;
a lead connector including a linear array of lead connector contacts each joined to a corresponding lead electrode via the assembly of elongated insulated conductors; the array of lead connector contacts including a selected contact corresponding with the first selected electrode;
a pulse generator device including a connector bore, the connector bore including a length and a device contact positioned along the length of the bore and adapted for electrical engagement of each of the array of lead connector contacts; and
means for reversibly locking the lead connector within the bore at multiple positions along the length of the bore; the multiple positions corresponding to electrical engagement of a different one of the array of lead connector contacts with the device contact;
wherein, at one of the multiple positions, the selected contact of the array of lead connector contacts is electrically engaged by the device contact.

2. The medical system of claim 1, wherein
the array of lead electrodes further includes a second selected electrode;
the array of lead connector contacts further includes a second selected contact corresponding to the second selected electrode; and
the connector bore further includes a second device contact, positioned adjacent to the device contact along the length of the bore; and
the second selected contact of the array of connector contacts is electrically engaged by the second device contact.

3. The medical system of claim 1, further comprising:
an additional electrode; and
an additional elongated insulated conductor; and
wherein the lead connector further includes a longer connector contact joined to the additional electrode, via the additional conductor, and positioned adjacent the array of connector contacts, the longer contact having a length approximately equal to a length of the array; and
the connector bore further includes a second device contact positioned adjacent the device contact along the length of the bore, the second device contact electrically engaging the longer connector contact at each of the multiple positions.

4. The medical system of claim 1, further comprising an insertion tool and wherein the connector bore further includes a proximal opening and a distal opening; the insertion tool adapted to be inserted into the proximal opening of the bore and to pull the lead connector through the distal opening of the bore and into the multiple positions.

5. The medical system of claim 1, wherein the means for reversibly locking the lead connector within the bore at the multiple positions along the bore includes a deflectable member projecting into the bore.

6. The medical system of claim 5, wherein:
each connector contact in the array of connector contacts includes a surface depression; and
the deflectable member is adapted to rest within the surface depression of each connector contact at each of the multiple positions.

7. The medical system of claim 5, wherein:
the linear array of lead connector contacts further includes a set of spacers, each spacer of the set of spacers separating each connector contact in the array of connector contacts and each spacer including a surface depression; and
the deflectable member is adapted to rest within the surface depression of each spacer at each of the multiple positions.

8. The medical system of claim 5, wherein:
the lead connector further includes an array of surface depressions positioned apart from the array of lead connector contacts; and
the deflectable member is adapted to rest within each surface depression of the array of surface depressions at each of the multiple positions.

9. The medical system of claim 1, wherein the means for reversibly locking the lead connector within the bore at the multiple positions along the bore includes an actuated member.

10. The medical system of claim 1, wherein the array of lead electrodes is substantially circumferential.

11. The medical system of claim 1, wherein the array of lead electrodes is substantially linear.

12. A method for selectively coupling a lead electrode, from an array of lead electrodes, to a pulse generator device, comprising:
positioning a lead connector, including an array of connector contacts corresponding to the array of lead electrodes, within a connector bore of the pulse generator device for electrical engagement of a selected connector contact, from the array of connector contacts, with a device contact positioned within the bore; the selected connector contact corresponding to the selected electrode.

13. The method of claim 12, wherein the step of positioning the lead connector comprises:
inserting an insertion tool into a proximal opening of the connector bore;
coupling the insertion tool to the lead connector; and
pulling the lead connector into the bore.

14. The method of claim 12, further comprising:
reversibly locking the lead connector in the electrically engaged position within the connector bore.

15. A method for directing electrical stimulation toward an epicardial surface of a heart comprising:
implanting a circumferential array of lead electrodes in a cardiac vein;
selecting one or more electrodes in contact with the epicardial surface of the heart from the array of lead electrodes;
positioning a lead connector including an array of connector contacts corresponding to the array of lead electrodes within a pulse generator connector bore such that one or more connector contacts corresponding to the one or more selected electrodes are electrically engaged by one or more device contacts positioned within the connector bore for electrical coupling of the selected one or more electrodes to the pulse generator.

16. The method of claim 15, wherein the step of positioning the lead connector comprises:
inserting an insertion tool into a proximal opening of the connector bore;
coupling the insertion tool to the lead connector; and
pulling the lead connector into the bore.

17. The method of claim 15, further comprising:
reversibly locking the lead connector in the electrically engaged position within the connector bore.

18. A method for selecting a pair of lead electrodes for electrical coupling to a pulse generator device, comprising:
implanting a linear array of lead electrodes; the array of lead electrodes including at least one electrode of the selected pair of lead electrodes;
positioning a lead connector, including an array of connector contacts corresponding to the array of lead electrodes, within a connector bore of the pulse generator device for electrical engagement of at least one selected connector contact from the array of connector contacts with a device contact; the at least one selected connector contact corresponding to the at least one electrode of the selected pair of lead electrodes.

19. The method of claim 18, wherein the step of positioning the lead connector comprises:
inserting an insertion tool into a proximal opening of the connector bore;
coupling the insertion tool to the lead connector; and
pulling the lead connector into the bore.

20. The method of claim 18, further comprising:
reversibly locking the lead connector in the electrically engaged position within the connector bore.
